(19) United States
(12) Patent Application Publication
(10) Pub. No.: US 2025/0072868 A1
(43) Pub. Date: Mar. 6, 2025

(54) ULTRASOUND DIAGNOSTIC APPARATUS AND IMAGE SELECTION METHOD

(71) Applicants: FUJIFILM Corporation, Tokyo (JP); NATIONAL CENTER FOR GLOBAL HEALTH AND MEDICINE, Tokyo (JP)

(72) Inventors: TAKESHI MATSUMURA, Tokyo (JP); Nobuyuki TAKEMURA, Tokyo (JP)

(73) Assignees: FUJIFILM Corporation, Tokyo (JP); NATIONAL CENTER FOR GLOBAL HEALTH AND MEDICINE, Tokyo (JP)

(21) Appl. No.: 18/817,188

(22) Filed: Aug. 27, 2024

(30) Foreign Application Priority Data
Aug. 29, 2023 (JP) 2023-139210

(51) Int. Cl. A61B 8/00; A61B 8/08

(52) U.S. Cl. CPC A61B 8/463; A61B 8/5292; A61B 8/54

(57) ABSTRACT

In a case in which a freeze operation is performed, an evaluation period is set for an image sequence in a cine memory based on first reference information. An image group belonging to the evaluation period is individually evaluated, and thus a recommended image is selected from the image group. The recommended image is displayed after the freeze operation.

### CROSS-REFERENCE TO RELATED APPLICATION

This application claims priority under 35 USC 119 from Japanese Patent Application No. 2023-139210, filed 29 Aug. 2023, the disclosure of which is incorporated by reference herein.

### BACKGROUND OF THE INVENTION

### 1. Field of the Invention

The present disclosure relates to an ultrasound diagnostic apparatus and an image selection method, and particularly relates to a technique of selecting an image to be displayed after a freeze operation.

### 2. Description of the Related Art

An ultrasound diagnostic apparatus is used in an ultrasonic examination of a subject. The ultrasound diagnostic apparatus is operated by an examiner (a doctor, an examination technician, or the like) who is a user.

In a state in which the ultrasound diagnostic apparatus performs a transmission/reception operation, that is, a real-time operation, a video image is displayed on a display of the ultrasound diagnostic apparatus. In a case in which an image of interest, such as a lesion part, appears during observation of the video image, the examiner performs the freeze operation. By the freeze operation, the transmission and the reception are stopped, and simultaneously with this, the display is switched from the video image to a still image. That is, the ultrasound diagnostic apparatus transitions from a real-time operation state to a freeze state. After the transition, the latest image acquired last is displayed as the still image. After that, the examiner searches for a desired image while performing an image rewinding operation or an image forwarding operation. In a case in which the desired image can be specified, the image is stored, and the image is incorporated into an examination report.

At a time point of transition to the freeze state, it is considered to perform control such that a past image acquired at a time point that is a predetermined period before the acquisition of the latest image or a past image acquired at the freeze operation time point is displayed instead of the latest image. However, such uniform processing reduces a likelihood that the displayed image is the image desired by the examiner.

In the ultrasound diagnostic apparatus disclosed in JP2012-161503A, a predetermined time range is determined based on a time of the freeze operation, and a diagnosis image is selected from among a plurality of images belonging to the predetermined time range. The predetermined time range is designated in advance by the user. JP2012-161503A does not disclose a technique of adaptively setting the predetermined time range.

The ultrasound diagnostic apparatus disclosed in JP2009-61040A has a voice input function. The pronunciation of “freeze” is recognized, and a freeze action is executed based on the recognition. In this ultrasound diagnostic apparatus, an image to be displayed after the transition to the freeze state is selected based on the start of voice input. JP2009-61040A does not disclose a technique of adaptively selecting the image to be displayed after the transition to the freeze state.

### SUMMARY OF THE INVENTION

An object of the present disclosure is to increase a likelihood that an appropriate image is displayed after a freeze operation. Alternatively, an object of the present disclosure is to appropriately determine a search range of an image to be displayed after the freeze operation.

An ultrasound diagnostic apparatus according to the present disclosure comprises: a storage unit that stores an image sequence consisting of a plurality of images arranged in time series, which are generated by repetition of transmission and reception of ultrasonic waves; a setting unit that adaptively sets an evaluation period for the image sequence based on reference information indicating a status of an ultrasonic examination in a case in which a freeze operation is performed; a selection unit that extracts an image group belonging to the evaluation period from the image sequence and evaluates each image constituting the image group to select a recommended image from the image group; and a display unit that displays the recommended image after the freeze operation.

An image selection method according to the present disclosure comprises: a step of storing, in a storage unit, an image sequence consisting of a plurality of images arranged in time series, which are generated by repetition of transmission and reception of ultrasonic waves; a step of adaptively setting an evaluation period for the image sequence based on reference information indicating a status of an ultrasonic examination in a case in which a freeze operation is performed; and a step of extracting an image group belonging to the evaluation period from the image sequence and evaluating each image constituting the image group to select a recommended image from the image group, in which after display of a video image based on the image sequence, the recommended image is displayed as a still image after the freeze operation.

According to the present invention, a likelihood that an appropriate image is displayed after a freeze operation is increased. Alternatively, a search range of an image to be displayed after the freeze operation is appropriately determined.

### BRIEF DESCRIPTION OF THE DRAWINGS

FIG. 1 is a block diagram showing an ultrasound diagnostic apparatus according to an embodiment.

FIG. 2 is a diagram showing freeze control (image selection method).

FIG. 3 is a diagram showing a configuration example of a freeze controller.

FIG. 4 is a diagram showing a first configuration example of a setting unit.

FIG. 5 is a diagram showing a second configuration example of the setting unit.

FIG. 6 is a diagram showing a first configuration example of a selection unit.

FIG. 7 is a diagram showing a second configuration example of the selection unit.

FIG. 8 is a diagram showing a display example.

### DESCRIPTION OF THE PREFERRED EMBODIMENTS

Hereinafter, an embodiment will be described with reference to the drawings.

### (1) Outline of Embodiment

An ultrasound diagnostic apparatus according to an embodiment includes a storage unit, a setting unit, a selection unit, and a display unit. The storage unit stores an image sequence consisting of a plurality of images arranged in time series, which are generated by repetition of transmission and reception of ultrasonic waves. The setting unit sets an evaluation period for the image sequence based on reference information indicating a status of an ultrasonic examination in a case in which a freeze operation is performed. The selection unit extracts an image group belonging to the evaluation period from the image sequence and evaluates each image constituting the image group to select a recommended image from the image group. The display unit displays the recommended image after the freeze operation.

With the above configuration, the evaluation period is set according to the status of the ultrasonic examination, and the recommended image is selected by individual evaluation of the image group belonging to which the evaluation period, so that a likelihood that the recommended image displayed after the freeze operation is an image desired by an examiner is increased. Since a series of processes after the freeze operation are automatically executed, no burden is generated on the examiner. After the display of the recommended image, the displayed image is manually switched as necessary. In this case, a likelihood of reaching the desired image in a short time is increased.

The above reference information is information referred to in order to set the evaluation period, and is information reflecting the current status of the ultrasonic examination. In the embodiment, the above reference information includes at least one of information for specifying a type of the image group (information for specifying an ultrasonic examination method), information for specifying the examiner, or information for specifying a type of the freeze operation. This reference information for setting the evaluation period is first reference information. Second reference information, which will be described below, is the same as the first reference information or is different from the first reference information. In the embodiment, the first reference information includes the second reference information.

In the embodiment, the setting unit includes a first determination unit and a second determination unit. The first determination unit determines a past reference time used as a reference in a case of setting the evaluation period, based on all or a part of the reference information. The second determination unit determines a time width that is a size of the evaluation period, based on all or a part of the reference information.

For example, one or both of the past reference time and the time width may be changed depending on the type of the image group, the examiner, or the type of the freeze operation. In a case in which one or both of the past reference time and the time width are changed based on a plurality of pieces of information, the evaluation period can be set more appropriately. The past reference time may be determined based on a time point at which a freeze command is issued or is recognized, a time at which a specific process is started or ended in response to the freeze command, or the like.

In the embodiment, the selection unit evaluates each image constituting the image group based on the second reference information indicating the status of the ultrasonic examination. For example, an evaluation method or an evaluation condition may be changed depending on the type of the image group or the examiner. In a case in which the image group is evaluated based on the plurality of pieces of information, a more appropriate evaluation result can be obtained.

The ultrasound diagnostic apparatus according to the embodiment further includes a generation unit. The generation unit generates a guide image including a first display element indicating the evaluation period and a second display element indicating a time point corresponding to the recommended image. The display unit displays the recommended image and the guide image. With this configuration, information indicating an acquisition timing of the displayed recommended image can be provided to the examiner, and information indicating the evaluation period in which the recommended image is searched for can be provided to the examiner. Through observation of the recommended image and the guide image, the examiner can determine necessity of a manual search, and can determine a direction and a range of the manual search in a case of performing the manual search.

An image selection method according to the embodiment includes a storage step, a setting step, and a selection step. In the storage step, an image sequence consisting of a plurality of images arranged in time series, which are generated by repetition of transmission and reception of ultrasonic waves is stored in the storage unit. In the setting step, in a case in which a freeze operation is performed, an evaluation period is set for the image sequence based on reference information indicating a status of an ultrasonic examination. In the selection step, an image group belonging to the evaluation period is extracted from the image sequence, and each image constituting the image group is evaluated. As a result, a recommended image is selected from the image group. After display of a video image based on the image sequence, the recommended image is displayed as a still image after the freeze operation.

The image selection method can be realized by a function of hardware or by a function of software. In the latter case, a program for executing the image selection method is installed in the ultrasound diagnostic apparatus via a network or a portable storage medium. The ultrasound diagnostic apparatus includes a non-transitory storage medium in which the program is stored.

### (2) Details of Embodiment

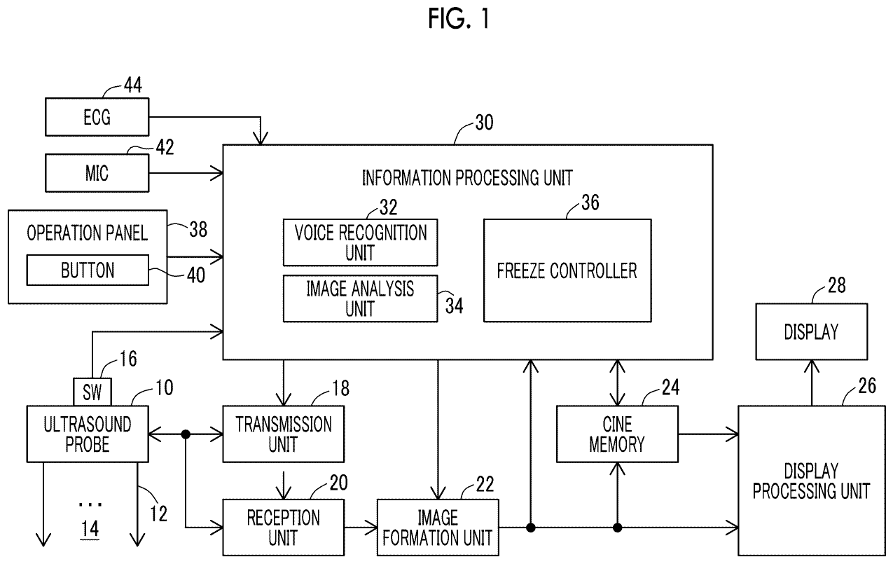

FIG. 1 shows the ultrasound diagnostic apparatus according to the embodiment. This ultrasound diagnostic apparatus is a medical apparatus used in an ultrasonic examination of a subject.

An ultrasound probe 10 is an instrument that transmits and receives ultrasonic waves. Specifically, the ultrasound probe 10 is composed of a probe head, a cable, and a connector. The probe head is held by the examiner, and a wave transmission/reception surface of the probe head is brought into contact with a surface of the subject. A vibration element array consisting of a plurality of vibration elements is provided in the probe head. The ultrasonic waves are emitted from the vibration element array into a living body, and reflected waves from the living body are detected by the vibration element array.

FIG. 1 shows an ultrasonic beam 12 formed by the vibration element array. A beam scanning surface 14 is formed by electronic scanning with the ultrasonic beam 12. The ultrasonic beam 12 corresponds to a transmission beam and a reception beam. As an electronic scanning method, an electronic linear scanning method, an electronic sector scanning method, or the like is known. A two-dimensional vibration element array may be provided in the probe head, and volume data may be acquired from an inside of the living body.

A switch 16 is provided on the probe head as necessary. The switch 16 is a freeze switch. A freeze signal is output to an information processing unit 30 by an operation of the switch 16. A probe that does not comprise the switch 16 may be used.

A transmission unit 18 is an electronic circuit that functions as a transmission beam former, and supplies a plurality of transmission signals to the vibration element array in parallel in a case of performing transmission. As a result, a transmission beam is formed.

A reception unit 20 is an electronic circuit that functions as a reception beam former, and applies phasing addition to a plurality of reception signals output in parallel from the vibration element array in a case of performing reception, thereby generating beam data. As the electronic scanning with the ultrasonic beam is performed, a plurality of pieces of beam data arranged in an electronic scanning direction are generated. The pieces of beam data constitute frame data. A beam data processing unit is provided between the reception unit 20 and an image formation unit 22, but the illustration thereof is omitted.

The image formation unit 22 forms an ultrasound image sequence (hereinafter, simply referred to as an image sequence) based on input frame data sequence. The image formation unit 22 includes a digital scan converter (DSC) as an electronic circuit. The DSC has a coordinate transformation function, a pixel interpolation function, and the like. The image sequence is composed of a plurality of images arranged on a time axis. Each image is, for example, a tomographic image (B-mode tomographic image). Various types of images are generated according to the ultrasonic examination method (including an operation mode). These images include, in addition to the tomographic image, a color flow mapping image (CFM image), an image including a puncture image (puncture image), an image including a contrast agent image (contrast image), and the like.

The image sequence generated by the image formation unit 22 is sent to a display processing unit 26 and is also sent to a cine memory 24. In a transmission/reception operation state, that is, a real-time operation state, the image sequence is displayed on a display 28 as a video image. In a freeze state, a still image read out from the cine memory 24 is displayed on the display 28. In the embodiment, after the freeze operation, first, an automatically selected recommended image is displayed. This will be described in detail below. The display 28 is configured of an organic EL display device, an LCD, or the like.

The cine memory 24 is a storage unit that stores the image sequence, and each image is temporarily stored in the cine memory 24. The cine memory 24 is configured of a semiconductor memory, a hard disk, or the like. The cine memory 24 has a ring buffer structure. A plurality of images output from the image formation unit 22 are sequentially stored in the cine memory 24 in time series. In the storage process, a process of overwriting and deleting the oldest image in the cine memory 24 is sequentially repeated.

A cine memory may be provided between the reception unit 20 and the image formation unit 22. In a case in which such a modification example is adopted, a frame data sequence is stored in the cine memory. The substance of the frame data sequence is an image sequence. It can be said that the image sequence is stored in the cine memory also in the modification example.

The information processing unit 30 functions as a controller and a calculation unit. In the embodiment, the information processing unit 30 is configured of a CPU that executes a program. The information processing unit 30 controls an operation of each component shown in FIG. 1. In FIG. 1, a part of a large number of functions exerted by the information processing unit 30 is represented by a plurality of blocks. The information processing unit 30 functions as a voice recognition unit 32, an image analysis unit 34, and a freeze controller 36.

An operation panel 38 is connected to the information processing unit 30. The operation panel 38 is an input device having a plurality of buttons, a plurality of knobs, a track ball, and the like. In FIG. 1, a freeze button 40 included in the operation panel 38 is shown. In a case in which the freeze button 40 is operated, the freeze signal is output from the freeze button 40 to the information processing unit 30.

A microphone 42 is provided as necessary. Specifically, the microphone 42 is provided in a case in which the operation of the ultrasound diagnostic apparatus is controlled by voice input. The voice recognition unit 32 applies voice recognition to a voice signal output from the microphone 42 and generates a command signal based on a result of the voice recognition. The information processing unit 30 executes control or processing in response to the command signal. In the embodiment, in a case in which the voice “freeze” is input to the microphone, the voice recognition unit 32 recognizes “freeze” and generates the freeze signal as the command signal.

The image analysis unit 34 functions in a case in which the operation of the ultrasound diagnostic apparatus is controlled by movement of the probe head. That is, the image analysis unit 34 functions in a case in which a gesture is input. Specifically, the image analysis unit 34 analyzes the input image sequence to analyze the type of the movement of the probe head. In the embodiment, retraction movement of moving the wave transmission/reception surface of the probe head away from a body surface of the subject is associated with the freeze operation. The image analysis unit 34 determines occurrence of the retraction movement based on the image sequence, and thus generates the freeze signal. The image analysis unit 34 is provided as necessary.

An electrocardiograph 44 is connected to the information processing unit 30. The electrocardiograph 44 generates an ECG signal. An electrocardiographic waveform is displayed on a screen of the display 28 based on the ECG signal. A foot pedal for the freeze operation may be connected to the information processing unit 30.

The freeze controller 36 executes freeze control of making transition from the ultrasound diagnostic apparatus from the real-time operation state to the freeze state in a case in which the freeze signal is generated. Specifically, in a case in which the freeze signal is generated, the freeze controller 36 stops the transmission and the reception, and simultaneously with this, switches the display from the video image to the still image. In the embodiment, the still image that is first displayed after the transition to the freeze state is a recommended image that is automatically selected. This will be described in detail below.

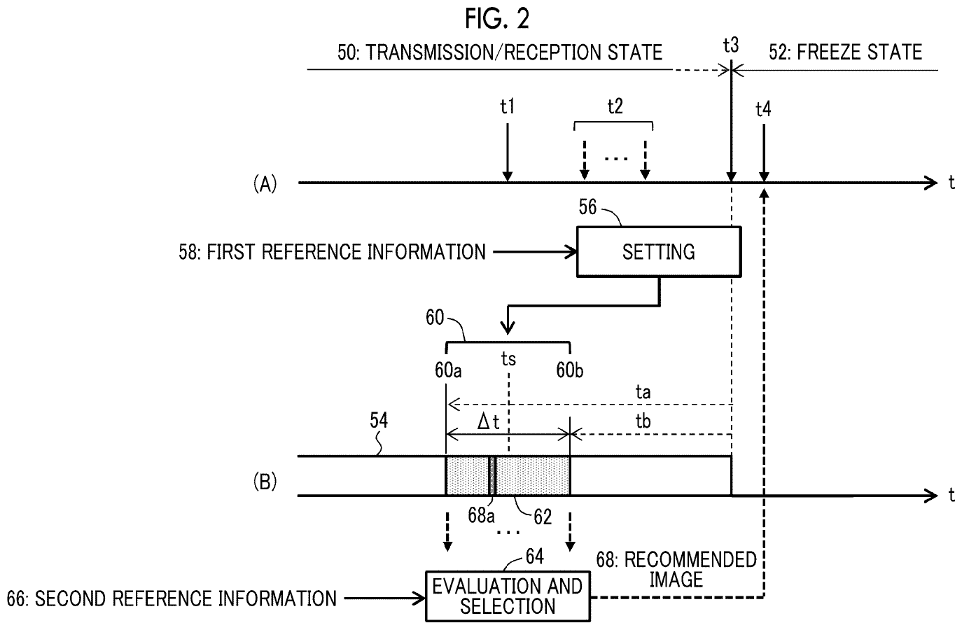

FIG. 2 schematically shows contents of the freeze control (image selection method) executed by the freeze controller. In FIG. 2, some details are exaggerated.
(A) shows a plurality of events on a time axis.
(B) shows a state of the cine memory.

In a video image observation state, for example, in a case in which a lesion part is recognized by the examiner at time point t1, the examiner performs the freeze operation at time point t2. A period (response period) from time point t1 to time point t2 is changed depending on the freeze operation method.

For example, in a case in which a method using a freeze button in the operation panel is selected, the response period is generally shortened. In a case in which a method using the voice input or a method using the probe operation is selected, the response period is generally lengthened. In a case of the method using the voice input, the response period greatly depends on an utterance speed. In a case in which a method using the switch provided in the probe is selected, the response period is changed depending on an examination site or a switch installation state. The response period is also changed by the examiner himself or herself. For example, in a case in which the examiner has a high ultrasonic examination skill, the response period is generally shortened. In a case in which the examiner has little experience in the ultrasonic examination, the response period is generally lengthened. The response period is also changed depending on the image type or the examination site.

In a case in which the freeze operation is recognized, the transition from the real-time state to the freeze state occurs as described above. The transition takes a certain amount of time until completion of the transition. Time point t3 indicates a time at which the freeze state is completed. The recommended image is displayed at time point t4. Time point t4 may coincide with time point t3. In the illustrated example, the state before time point t3 is the transmission/reception state (real-time operation state). The state after time point t3 is the freeze state.

In a case in which the freeze operation is performed at time point t2, the freeze controller sets an evaluation period (evaluation section) 60 for the image sequence 54 stored in the cine memory based on first reference information 58 (see reference numeral 56).

Specifically, the freeze controller determines the past reference time ts based on the first reference information 58, and determines a time width Δt based on the first reference information 58. In the embodiment, a center of the evaluation period is determined by the past reference time ts, and a time length of the evaluation period is determined by the time width Δt.

Information (time stamp) indicating a time at which each image is acquired (or generated) is added to each image. The information may be used in specifying the image group. The past reference time ts may be determined by using a time at which the freeze command is issued, a time at which the freeze command is recognized, a predetermined time in the freeze control process, or the like, as a reference time for calculation. A time length ta and a time length tb for defining the start and the end of the evaluation period 60 may be determined by using time point t3 as a reference for calculation.

The first reference information is information referred to in a case of setting the evaluation period. For example, the first reference information can include information for specifying the examiner, information for specifying the type of the image sequence (which may also be information for specifying the examination method or information for specifying the examination site), information for specifying the freeze operation method, and the like. Specific examples of the first reference information will be described below.

The freeze controller specifies an image group 62 belonging to the evaluation period 60 in the image sequence stored in the cine memory, and reads out the image group 62 from the cine memory. Subsequently, the freeze controller individually evaluates a plurality of images constituting the image group 62 based on the second reference information, and assigns a rank (which can also be referred to as a score or an evaluation value) to each image (see reference numeral 64). The freeze controller specifies the highest rank from among a plurality of the ranks assigned, and selects an image 68a to which the highest rank is assigned as a recommended image 68 (see reference numeral 64).

The second reference information is information referred to in a case of evaluating each image constituting the image group belonging to the evaluation period. For example, the second reference information can include information for specifying the examiner, information for specifying the type of the image sequence, and the like. All or a part of the first reference information may be the second reference information. Specific examples of the second reference information will be described below. Various known methods can be used as an image evaluation method. The image evaluation method or an image evaluation condition may be switched depending on the type of the image sequence. An image evaluation model may be switched depending on the type of the image sequence.

The freeze controller displays the recommended image selected as described above on the display. The display timing is time point t4, and it is desired to execute the freeze control such that time point t4 approaches time point t3 as close as possible, and ideally, time point t4 matches time point t3. The recommended image is a still image first displayed after the freeze operation. Thereafter, the displayed image is switched as necessary. In this case, an image forwarding operation or an image rewinding operation is performed starting from the image 68a. The image search may be performed beyond the image group 62.

According to the above-described freeze control, the evaluation period is set according to the status of the ultrasonic examination, and the image group can be individually evaluated in consideration of the status of the ultrasonic examination, so that after the freeze operation, a likelihood that the image desired by the examiner is displayed as the recommended image is increased. No burden is generated on the examiner in a case of displaying the recommended image. Even in a case in which the recommended image is not the image desired by the examiner, a likelihood of performing the image search in a short time can be increased.

The examiner can select whether or not to execute the freeze control. In a case in which the non-execution of the freeze control is selected, the freeze control in the related art is executed. In this case, the latest image acquired last after the transition to the frozen state is displayed as the still image. The freeze control method may be switched by switching an operation aspect of the input device for performing the freeze input. For example, in a case in which the freeze button provided on the operation panel is pressed for a normal time (short time), the freeze control in the related art may be executed, and in a case in which the freeze button is pressed for a long time, the above-described freeze control may be executed. A switch, a button, or the like for switching the freeze control method may be provided.

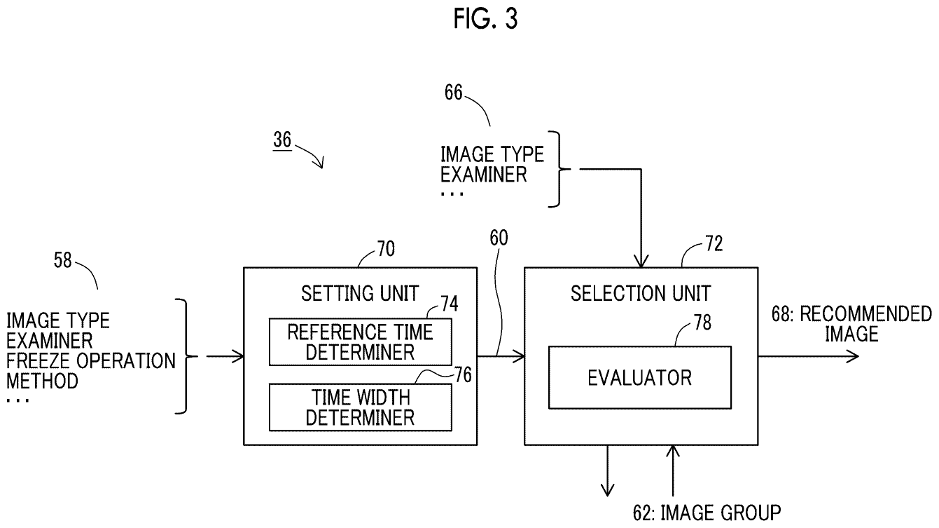

FIG. 3 shows a configuration example of the freeze controller 36. The freeze controller 36 consists of a setting unit 70 and a selection unit 72. The setting unit 70 includes a reference time determiner 74 that functions as a first determination unit and a time width determiner 76 that functions as a second determination unit. The reference time determiner 74 determines the past reference time ts based on all or a part of the first reference information 58. The time width determiner 76 determines the time width Δt based on all or a part of the first reference information 58. The evaluation period 60 is determined using the past reference time ts and the time width Δt.

FIG. 3 shows a specific example of the first reference information 58. The first reference information 58 includes information for specifying the image type (type of the image sequence), information for specifying the examiner, information for specifying the freeze operation method, and the like. The first reference information 58 may include information for specifying a frame rate, information for specifying a cardiac cycle, information for specifying a time required for the freeze operation (or the freeze input), and the like.

The selection unit 72 is notified of the evaluation period 60 set by the setting unit 70. The selection unit 72 reads out the image group 62 from a frame memory. The selection unit 72 includes an evaluator 78 that individually evaluates the image group 62. The evaluator 78 evaluates each image based on second reference information 66. The second reference information 66 includes, for example, information for specifying the image type and information for specifying the examiner. The second reference information 66 may include other information. The evaluator 78 assigns a rank to each image. The selection unit 72 selects an image to which the highest rank is assigned as the recommended image 68.

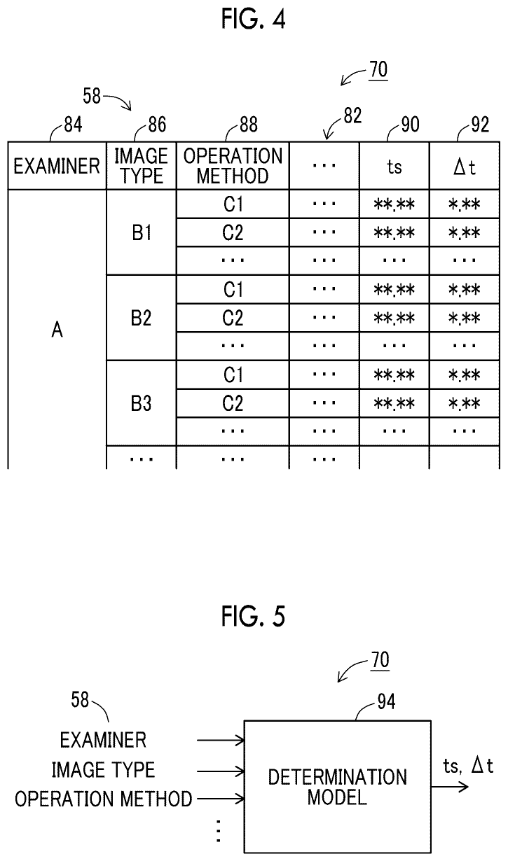

FIG. 4 shows a first configuration example of the setting unit 70. The setting unit 70 includes a look-up table (LUT) 82. The LUT 82 functions as the reference time determiner and the time width determiner described above. An LUT for the reference time determiner and an LUT for the time width determiner may be provided.

In the LUT 82, a past reference time (ts) 90 and a time width (Δt) 92 are registered for each combination of an examiner 84, an image type 86, a freeze operation method, and the like. That is, in a case in which the combination specified by the first reference information 58 is input to the LUT 82, the past reference time 90 and the time width 92 corresponding to the combination are output from the LUT.

FIG. 5 shows a second configuration example of the setting unit 70. The setting unit 70 includes a determination model 94 that functions as the reference time determiner and the time width determiner. The determination model 94 is generated by machine learning. For example, the determination model 94 is composed of a network such as a CNN. In a case in which the information for specifying the examiner, the information for specifying the image type, the information for specifying the freeze operation method, and the like are given as the first reference information 58 to the determination model 94, the determination model 94 outputs information for specifying the past reference time ts and the time width Δt that are suitable for the first reference information 58. A determination model that determines the past reference time and a determination model that determines the time width may be provided.

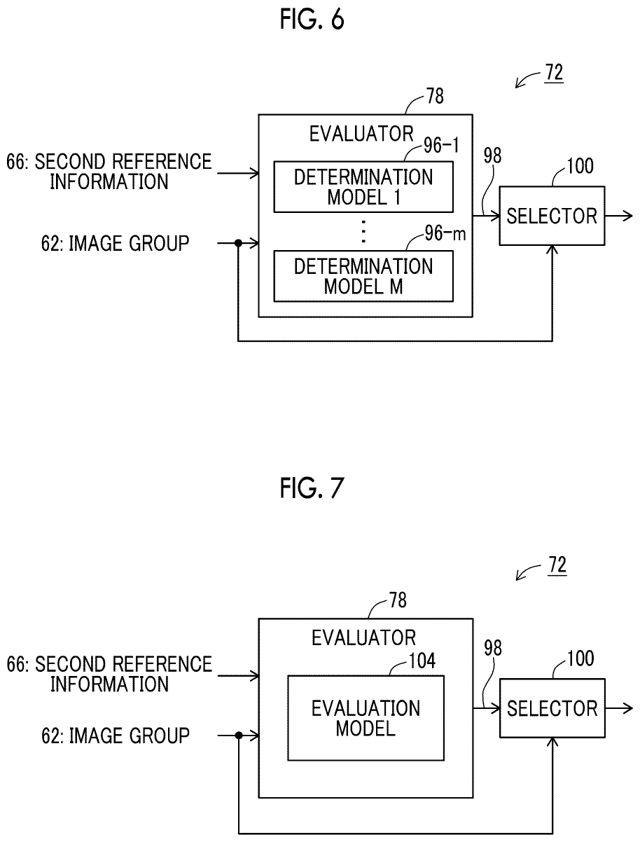

FIG. 6 shows a first configuration example of the selection unit 72. The selection unit 72 includes the evaluator 78 and a selector 100. The second reference information 66 and the image group 62 are input to the evaluator 78. The evaluator 78 includes a plurality of evaluation models 96-1 to 96-m. Each of the evaluation models 96-1 to 96-m is an image evaluation model generated by machine learning. An evaluation model to be actually used is selected based on the second reference information 66. The image group 62 is given to the evaluation model. The evaluation model evaluates superiority or inferiority of each image and outputs a rank 98 for each image. The evaluator 78 is composed of a network such as a CNN.

For example, the plurality of evaluation models 96-1 to 96-m correspond to a plurality of image types. Alternatively, the plurality of evaluation models 96-1 to 96-m correspond to a plurality of combinations (a combination of the image type and the examiner).

The selector 100 receives a plurality of the ranks 98 generated by the evaluation of the plurality of images. The selector 100 specifies the highest rank among the plurality of ranks 98 and selects an image corresponding to the highest rank from the image group 62 as the recommended image.

For example, in a case of evaluating the tomographic image, clarity of a tissue image may be evaluated. In a case of evaluating a blood flow image (including a CFM image), clarity of the blood flow image or a size of the blood flow image may be evaluated. In a case of evaluating the puncture image, clarity of a puncture needle image may be evaluated. In a case of evaluating the contrast image, clarity of a contrast agent image or a size of the contrast agent image may be evaluated.

FIG. 7 shows a second configuration example of the selection unit 72. In FIG. 7, the same reference numerals are given to the same components as those of the components shown in FIG. 6, and the description thereof will be omitted. The evaluator 78 includes a single evaluation model 104. The evaluation model 104 evaluates each image constituting the image group 62 while taking into consideration the second reference information 66. The evaluation model 104 is also generated by machine learning. The evaluation model 104 is composed of a network such as a CNN.

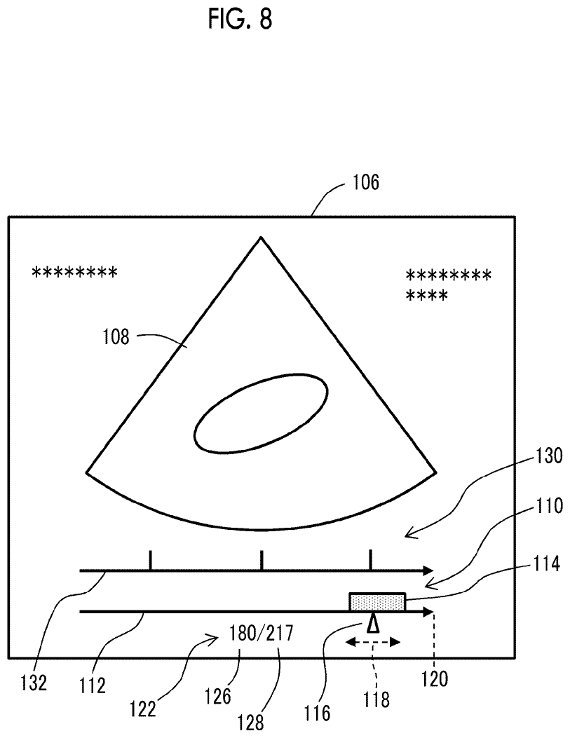

FIG. 8 shows an example of an image displayed in a case in which the freeze state is established. A recommended image 108 is displayed on a screen 106 of the display. In the illustrated example, the recommended image 108 is a tomographic image. The CFM image, the puncture image, the contrast image, and the like are displayed depending on the ultrasonic examination method.

An electrocardiographic waveform 130 is displayed below the recommended image 108. The electrocardiographic waveform 130 includes a FIG. 132 indicating a time axis and a plurality of R-waves. A guide image 110 is displayed below the electrocardiographic waveform 130. The guide image 110 includes a FIG. 112 indicating a time axis, a FIG. 114 showing an evaluation period, and a FIG. 116 showing a time of the acquisition of the recommended image. A right end 120 of the time axis corresponds to a time of the establishment of the freeze state. A time axis scale of the electrocardiographic waveform 130 matches a time axis scale of the guide image 110. Each figure is a display element.

The start and the end of the evaluation period can be easily recognized through observation of the FIG. 114. The time of the acquisition of the recommended image can be easily recognized through observation of the FIG. 116. Although the FIG. 114 has a band-like form, another form may be adopted instead of the band-like form. Although the FIG. 116 has a triangular shape, another form may be adopted instead of the triangular shape.

An indicator 122 is composed of a numerator 126 and a denominator 128. The denominator 128 represents the total number of images constituting the image sequence stored in the cine memory. The numerator 126 indicates the number of the recommended image 108 in the total number of images.

After the display of the recommended image 108, in a case in which the image forwarding operation and the image rewinding operation are performed, the FIG. 116 slides in a right direction or a left direction (see reference numeral 118). As a result, the images displayed on the screen 106 are sequentially switched. The content of the indicator 122 is also changed. With the guide image 110, the examiner can intuitively recognize a time at which the recommended image is acquired, a size and a position of the evaluation period, and the like.

A plurality of the recommended images may be displayed after the freeze operation. In this case, a plurality of recommended images that are consecutive in time may be displayed, or a plurality of recommended images that are discretely specified on a time axis may be displayed. A graph showing a temporal change of a rank sequence assigned to the image sequence may be displayed in a figure (box) 114. In a case in which the configuration is adopted, the image can be easily searched for. Such a graph may be displayed outside the FIG. 114.

What is claimed is:

1. An ultrasound diagnostic apparatus comprising:
a storage unit that stores an image sequence consisting of a plurality of images arranged in time series, which are generated by repetition of transmission and reception of ultrasonic waves;
a setting unit that sets an evaluation period for the image sequence based on reference information indicating a status of an ultrasonic examination in a case in which a freeze operation is performed;
a selection unit that extracts an image group belonging to the evaluation period from the image sequence and evaluates each image constituting the image group to select a recommended image from the image group; and
a display unit that displays the recommended image after the freeze operation.

2. The ultrasound diagnostic apparatus according to claim 1,
wherein the setting unit includes
a first determination unit that determines a past reference time used as a reference in a case of setting the evaluation period, based on all or a part of the reference information, and
a second determination unit that determines a time width that is a size of the evaluation period, based on all or a part of the reference information.

3. The ultrasound diagnostic apparatus according to claim 1,
wherein the reference information includes at least one of information for specifying a type of the image sequence, information for specifying an examiner, or information for specifying a type of the freeze operation.

4. The ultrasound diagnostic apparatus according to claim 1,
wherein the reference information is first reference information, and
the selection unit evaluates each image constituting the image group based on second reference information indicating the status of the ultrasonic examination.

5. The ultrasound diagnostic apparatus according to claim 4,
wherein the second reference information includes at least one of information for specifying a type of the image sequence or information for specifying an examiner.

6. The ultrasound diagnostic apparatus according to claim 1, further comprising:
a generation unit that generates a guide image including a first display element indicating the evaluation period and a second display element indicating a time point corresponding to the recommended image,
wherein the recommended image and the guide image are displayed on the display unit.

7. An image selection method comprising:
a step of storing, in a storage unit, an image sequence consisting of a plurality of images arranged in time series, which are generated by repetition of transmission and reception of ultrasonic waves;
a step of setting an evaluation period for the image sequence based on reference information indicating a status of an ultrasonic examination in a case in which a freeze operation is performed; and
a step of extracting an image group belonging to the evaluation period from the image sequence and evaluating each image constituting the image group to select a recommended image from the image group,
wherein after display of a video image based on the image sequence, the recommended image is displayed as a still image after the freeze operation.
